(19) United States
(12) Patent Application Publication
(10) Pub. No.: US 2025/0244302 A1
(43) Pub. Date: Jul. 31, 2025

(54) Method and Device for Determining the Amount of Adsorption of Sorbents in at Least One Storage Substance as Well as a Computer Program Product

(71) Applicant: Robert Bosch GmbH, Stuttgart (DE)

(72) Inventors: Philipp Nolte, Gerlingen (DE); Helmut Schmidt, Bietigheim Bissingen (DE); Ulrike Mock, Pforzheim (DE); Anne Fuchs, Gerlingen (DE)

(21) Appl. No.: 19/029,804

(22) Filed: Jan. 17, 2025

(30) Foreign Application Priority Data
Jan. 31, 2024 (DE) 10 2024 200 865.6

(51) Int. Cl. G01N 33/00

(52) U.S. Cl. CPC G01N 33/004

(57) ABSTRACT

A method for determining the amount of adsorption of sorbents in at least one storage substance, includes determining the amount of adsorption of the sorbents taking into account both physical and chemical parameters of the at least one storage substance and taking into account a theoretical and/or data-based adsorption behavior of the at least one storage substance based on a plurality of physical data associated with different points in time and/or periods of time.

This application claims priority under 35 U.S.C. § 119 to application no. DE 10 2024 200 865.6, filed on Jan. 31, 2024 in Germany, the disclosure of which is incorporated herein by reference in its entirety.

### FIELD

The disclosure relates to a method for determining the amount of adsorption of sorbents in at least one storage substance, which may be used in particular in connection with the removal of CO₂ from the atmosphere and the storage of the CO₂ in storage substances. Further, the disclosure relates to a device, in particular to a data processing system for carrying out the method, as well as a computer program product.

### BACKGROUND

As climate change advances, possible approaches are being sought more and more to reduce the increase in CO₂ in the atmosphere or to remove the CO₂ present in the atmosphere and store it in suitable storage media. The calculation methods used for this purpose are typically described by simple sorption models using experimental laboratory data and a constant or fixed temperature. Such computational methods, which are designed to be relatively simplified, appear to have a potential for improvement in terms of their accuracy with regard to the complexity of the processes taking place.

### SUMMARY

The method according to the disclosure for determining the amount of adsorption of sorbents in at least one storage substance disclosed herein has the advantage that it calculates the amount of adsorption more realistically using a theoretical and/or data-based adsorption behavior based on a carrier medium containing the sorption medium and thereby allows for far more accurate information to be provided regarding the expected amount of adsorption of the sorbent. This is because, in contrast to the methods known from the prior art, constant parameters of the carrier medium are not used as the basis, but rather parameters that prevail at different times and/or periods.

In particular, the method according to the disclosure is thus also suitable for the assessment/selection of possible locations for corresponding systems for storing CO₂, because location-specific weather data is taken into account for the air as the carrier medium for the CO₂.

In light of the above explanations, the method according to the disclosure for determining the amount of adsorption of sorbents in at least one storage substance has the features that, taking into account both physical and chemical parameters of the at least one storage substance and taking into account a theoretical and/or data-based adsorption behavior of the at least one storage substance, the amount of adsorption of the sorbent is determined by means of a plurality of physical data of a carrier medium associated with different points in time and/or periods of time, wherein the physical data comprises at least a temperature of the carrier medium that contains the sorbent and that chemically reacts with or is configured to react chemically with the at least one storage substance at different times and/or periods of time.

The method according to the disclosure is preferably used to determine the amount of adsorption of CO₂ as a sorbent, wherein the physical data of the carrier medium is the weather data of a location and wherein the weather data comprises at least the air temperature.

The method has a particularly high accuracy when additionally considering a pressure of the carrier medium at the different times and/or time periods.

With respect to weather data, it is possible on the one hand to use historical weather data from weather services. However, additionally or instead of this, forecasted weather data may also be used, which relates in particular to an amount of time that CO₂ storage is to be carried out at a potential location. This may be particularly useful when forecasting future heating/climate changes for a location.

In furtherance of the most recent suggestion, it may be contemplated that weather data evaluated or forecasted at certain times and/or periods may be used to determine the amount of adsorption. For example, in particular, the amounts of the sorbent adsorbed at different times of year and/or day may be determined and evaluated.

This may also be used to compare different locations with respect to possible amounts of adsorption, wherein location-specific weather data for each location is used.

The following explanation is provided with regard to weather data: The data set for the weather at a location can be based on own measurements or databases (e.g., of the German weather service). Ideally, it includes temperature, humidity and air pressure together with a timestamp. If the air pressure is not available, an approximate partial CO₂ pressure which is independent of the weather, e.g. 0.4 mbar, can also be used. If a continuous deviation or a deviation changing over time of the CO₂ content from the natural average is to be assumed for the location, then data from sensors, satellite imagery or modeling data can also be used. If geometry and operating parameters are also known for a CO₂ storage system, for instance, the weather data can additionally be pre-processed, e.g. an increased dynamic pressure at the inlet of the system in calculating the partial CO₂ pressure or an air temperature deviating from the ambient temperature at the location of the sorbent.

At high temperatures and humidity levels (e.g. above 50° C.), in order to obtain particularly precise values for the molar fraction or the amount of material of CO₂ it is recommended to use a dilution factor of the CO₂ by the water content in the air (e.g., an absolute humidity of 4% in the air leads to a reduction of 400 ppm CO₂ to only 400/1.04 ppm). Conversely, to accelerate the calculation this exact step can be omitted. To accelerate the calculation, the model may be accelerated by numeric approximations. For example, the complete model may be provided on a sampling point grid consisting of temperature and humidity with a specific grid design. A numerically simple, fitted model (e.g., 1st-3rd degree polynomial model, machine learning model) may be used for approximation or interpolation between these support points.

If different possible locations are to be compared or evaluated with respect to their suitability for CO₂ storage, the method on the one hand provides for using location-specific weather data, on the other hand, however, for reasons of simplicity, it provides for assuming fixed physical and chemical parameters of the at least one storage substance.

With regard to the calculation of the amount of adsorption, it provides in particular in connection with CO₂ as a sorbent that the amount of adsorption is calculated based on static physics, wherein the proportions for how different possible adsorbents of the at least one storage substance and empty spaces are distributed across uniform receptors are calculated.

Furthermore, the method according to the disclosure is used to depict the thermodynamically possible filling state between 0% and 100% of a storage material with CO₂, depending on the ambient temperature and humidity. Kinetic aspects are not considered, so the physical model yields the maximum possible fill level in the favorable case.

In further development of the method just described, it is provided that for each possible adsorbent, the at least one storage substance will take into consideration the stoichiometry of all involved sorbents, calculate the entropy of the receptors, and link the chemical potentials of the storage substance for each adsorbent to the chemical potentials of the gases involved in the process.

It is also envisioned in a further development of the method that sorbents are considered which are chemically bound by more than one receptor of the adsorbing materials of the at least one storage substance.

In a further development of the proposal just described, it may be contemplated that the distances and spatial arrangement of the receptors are grouped together into individual cluster types and that the cluster types are considered as receptors.

By means of the method according to the disclosure, windows of time (e.g., certain months in the year or certain times) in which a particularly high or low amount of storage is to be expected can be predicted, as well as whether the storage amounts are uniform or fluctuating.

It is further explained that the method generally described herein may be used not only for determining or calculating the storage of CO₂, but also for other substances or sorbents and for other technical equipment in the area of filtering. The method according to the disclosure may also be used for liquid chemical or solutes in liquids.

Furthermore, the disclosure comprises a device, in particular a data processing system configured to perform a method according to the disclosure.

Lastly, the disclosure also comprises a computer program product, in particular a data program or a data storage medium, comprising commands that cause the device to perform at least one of the method steps according to the disclosure.

Further advantages, features, and details of the disclosure will emerge from the following description of embodiments of the disclosure and from the drawings.

### BRIEF DESCRIPTION OF THE DRAWINGS

FIG. 1 and FIG. 2 show flowcharts for explaining possible approaches for location selection for systems for storing sorbents based on weather data;

FIG. 3 depicts an illustration of a thermodynamic model system consisting of a gas phase and sorbent;

FIG. 4 depicts a diagram of the CO₂ occupancy of receptors at different assumed binding energies over the temperature profile;

FIG. 5 and FIG. 6 show diagrams of the CO₂ occupancy of the receptors at different binding energies and different partial pressures;

FIG. 7 shows a diagram of occupancy at different partial CO₂ pressures at a defined water vapor partial pressure;

FIG. 8 shows an illustration of different receptors in irregular spatial arrangement that are combined and categorized as clusters; and

FIG. 9a and FIG. 9b show illustrations of successive steps for modeling adsorption when two receptors are involved for formation in one of the adsorbing materials.

### DETAILED DESCRIPTION

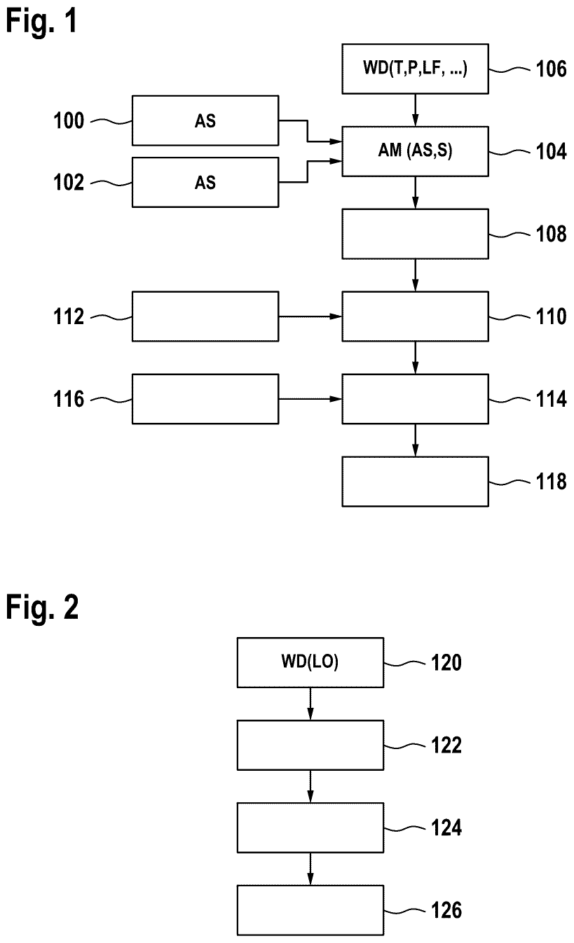

In FIG. 1, a basic method for linking data with respect to determining an amount of adsorption AM of CO₂ as sorbent S in at least one storage substance or adsorbing material AS is shown in conjunction with consideration of weather data WD in the form of a flow chart.

In FIG. 1, block 100 comprises data that takes into account chemical parameters of possible adsorbing materials AS and stoichiometries. Block 102 comprises data for the physical input parameters of possible adsorbing materials AS. The physical input parameters comprise, in particular, binding energies or enthalpies, wherein the input parameters indicated are either known or available as assumed input parameters.

The data from the two blocks 100, 102 are fed to a block 104 as input variables. Block 104 comprises a computer program as part of a data processing system, wherein the computer program comprises an algorithm that enables determination of the amount of the assumption AM as a function of the adsorbing material or materials AS and the sorbents S.

Weather data WD is fed to the block 104 via a block 106 as an input variable. In the context of the disclosure, weather data WD means weather data WD of a possible (geographical) location LO of a system for storing the sorbents S, in particular for CO₂ storage. The weather data WD may be either historical weather data WD, or forecasted weather data WD (for the future). The weather data WD comprises, for example, in particular over longer periods of time, daily recorded weather data WD over several years comprising at least the air temperature T and the air pressure P. Preferably, the weather data WD also comprises the humidity LF. The air thereby represents a carrier medium for the CO₂ in the atmosphere.

For example, in block 104, the algorithm determines amounts of adsorption AM for each day for which the weather data is available. For example, these amounts of adsorption AM can be summed up for certain periods of time, and average values can be formed or evaluated statistically. This takes place in block 108.

The data obtained in block 108 is then further processed in a block 110. Block 110 is used to agglomerate the determined data using a fixed data filter. To this end, block 110 is provided with data as inputs via block 112 that comprises technical or economic criteria for operating a system for removing CO₂ from the atmosphere.

The data obtained in block 110 may then be further processed in a block 114. Block 114 represents a second data filter and is used for further bundling and Evaluation of the data. For this purpose, data is fed to block 114 via a block 116, which serves to evaluate the classification for achieving target values based on technical and economic considerations. The data obtained in block 114 is then illustrated or output in a block 118, wherein a degree of achievement of performance objectives is documented in block 118. The blocks 100 to 118 described above can be realized in the form of a computer program product in the data processing system or on a computer.

FIG. 2 illustrates a method for evaluating or assessing different locations LO based on the basic method discussed in connection with FIG. 1. The method according to FIG. 2 comprises a block 120 containing data sets of weather data WD from various possible and potential locations LO (which may be located in different climate zones). The data sets present in block 120 as an input variable are then subjected to an evaluation method in a block 122 with the same or fixed physical and chemical parameters (of the sorbents S as well as the adsorbents AS), respectively. The block 124 represents an output block that contains a level of achievement of performance targets for each material variant. Lastly, in a block 126, a ranking of locations LO and climate zones is generated.

Taken together, the methods shown in FIG. 1 and FIG. 2 serve to select or compare suitable locations LO for systems for the removal of in particular CO₂ as a sorbent S from the atmosphere serving as a carrier medium.

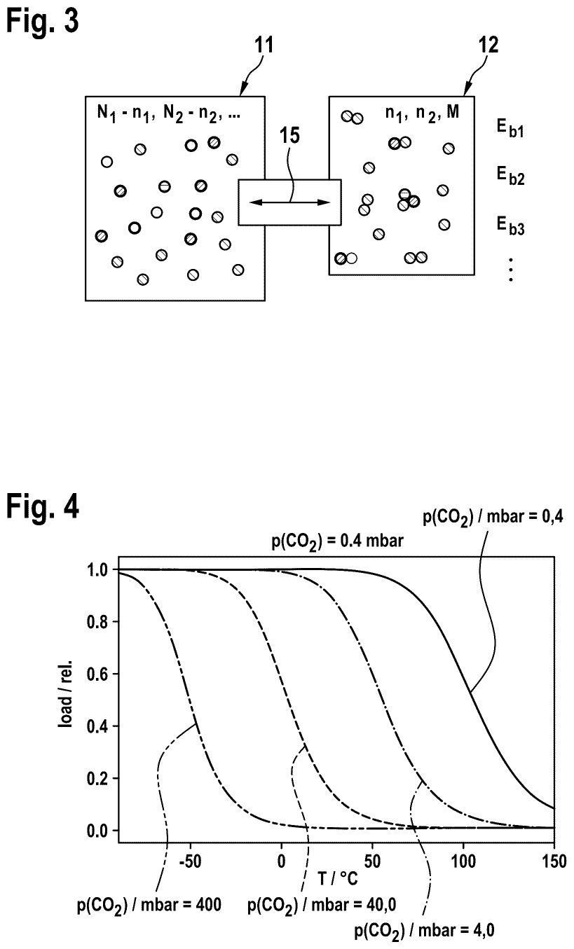

With regard to the determination or calculation of adsorption amounts AM of sorbents S from at least one storage substance or an adsorbent AS, FIG. 3 is initially referred to in the following. In FIG. 3, the stored gas quantity in an adsorbent AS is shown from a thermodynamic point of view. A first sub-system 11 shown on the left side of FIG. 3 depicts the gas phase, which can consist of several gas varieties of different concentrations (e.g. nitrogen, oxygen, CO₂, water). There is a sorbent with the same kinds of receptors in the second sub-system 12 shown on the right side of FIG. 3, wherein the two sub-systems 11, 12 interact, as is illustrated by the double arrow 15. Some of the gases may be attached to each receptor n₁, n₂, etc. However, there are only discrete ways they can be occupied by one or more of the gases. Each variant is referred to as an adsorbent AS. The adsorbent AS is assigned a binding energy E_{b,i} or adsorption enthalpy ΔHᵢ. There may be k different adsorbents AS. Not all gas types need to be involved in adsorbing (e.g. oxygen, nitrogen). The receptors may remain unfilled.

In order to determine the adsorption amount AM, it is first necessary to clarify which proportion of the receptors is unfilled and which proportion is occupied with the adsorbent types i=1.2, . . . k, namely depending on temperature T and depending on the initial number of all molecules of each gas type in the gas phase. According to the laws of thermodynamics, the equilibrium condition is that the gas molecules must be distributed between the gas phase and the sorbent such that the free energy F=E−TS is minimal. Here, E is the total energy of both sub-systems 11, 12, S is the total entropy of both sub-systems 11, 12. The free energy F may be calculated individually for both sub-systems 11, 12 and equated to the particle flow direction taking into account the signs.

If there is a large gas reservoir with a practically fixed concentration (despite adsorption), it is practical to use Gibbs free energy G (pi, T)=F+pV and to calculate using concentrations of the gases rather than particle numbers. If a volume change is neglected for the sorbent (at least a volume increase will be significantly lower than the volume the ab/adsorbed sorbent S would have taken up in a gas form), then no further differentiation between free energy and Gibbs free energy must be considered for it in the equations.

As the equilibrium condition, the derivation of F (or G) must be equated with the corresponding negative derivatives regarding the gases (left sub-system 11) in relation to all adsorbents AS in the sorbent (right sub-system 12). In addition, stoichiometric coefficients must be considered, because not every adsorbent AS must be composed of exactly one substance.

Using the example of two gases A and B and k different adsorbents AS, a system of k reaction equations results.

$${v}_{1 A} \cdot A + {v}_{1 B} + Recept ⇋ {Adsorbent}_{1} , \\ enthalpy of Δ {H}_{1} ( T ) or binding energy \cdot {E}_{b 1} \qquad ( 1 )$$

$${v}_{2 A} \cdot A + {v}_{2 B} + Recept ⇋ {Adsorbent}_{2} , \\ enthalpy of Δ {H}_{2} ( T ) or binding energy \cdot {E}_{b 2} \qquad ( 2 )$$

$$⋮$$

$${v}_{kA} \cdot A + {v}_{kB} + Recept ⇋ {Adsorbent}_{k} , \\ enthalpy of Δ {H}_{k} ( T ) or binding energy \cdot {E}_{k 1} \qquad ( k )$$

In the sorbent, the energy can be balanced as a weighted sum of all binding energy with the respective occupations: E_{sorb}=m₁·E₁−m₂·E₂ . . . −mₖ·Eₖ. The entropy or binding energy: ia S_{sorb}=k_{B}. In(Ω). In this respect, k_{B} is the Boltzmann constant and Ω is the number of possible micro-states that an occupancy with m₁,m₂, . . . mₖ adsorbents AS of types 1, 2, . . . k can fulfill. The number of unoccupied receptors is necessarily determined to be (M−m₁−m₂ . . . mₖ):

$$Ω ( M , {m}_{1} , ... , {m}_{k} ) = \frac{m !}{{m}_{1} ! {m}_{2} ! ... {m}_{k} ! ( M - {m}_{1} - {m}_{2} ... - {m}_{k} ) !}$$

The k chemical potentials of adsorbents AS are derived from the partial derivatives of the free energy/Gibbs free energy of the sub-system of the sorbent according to m₁, m₂, . . . mₖ. These must be linked to the derivatives of the free energy of the gases (or the chemical potentials) of the gases. The stoichiometric factors vᵢⱼ must be taken into account.

The temperature-dependent chemical potentials of the gases can be determined from the relevant literature tables for gases. If the gases do not behave ideally, these properties are in the referenced chemical potentials.

Finally, from the k equations, the relative fraction αᵢ=m√M) at the receptors can be determined for each adsorbent AS and the empty spaces if the gas concentrations and the temperatures T are known.

FIG. 4 shows a diagram with a first example of an ideal selective sorbent that binds only one gas type (here: CO₂) and no further gas, wherein exactly one CO₂ molecule is bound per receptor. There is competition from occupied receptors and non-occupied receptors. The temperature profiles of the relative CO₂ occupancy (load/rel.) of the receptors are overlaid in FIG. 4, namely for a partial CO₂ pressure of 0.4 mbar (corresponding to 400 ppm at 1 bar total pressure) at different assumed binding energy levels (Bb/eV). At high and low temperatures T, the curves approach asymptotically horizontal lines and show a sharp slope in a transition region that has a nearly linear curve with temperature T between approximately 20% and 80%. These slopes change slightly with the binding energy Bb/eV and they become flatter the weaker the binding energy Bb/eV is.

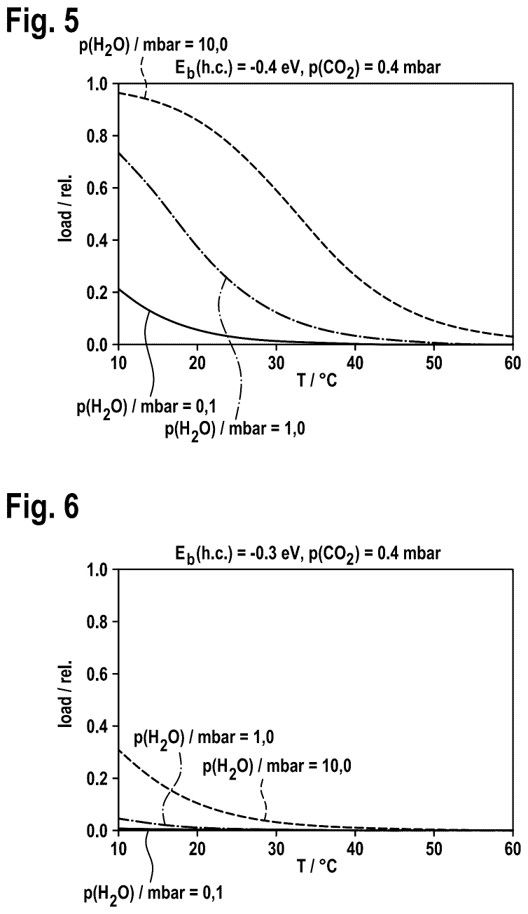

FIGS. 5 and 6, as a second example, show a further ideal selective sorbent that binds exactly one composite adsorbent per formulation, consisting of one H₂O molecule and on CO₂ molecule. The upper and lower diagrams differ by the binding energy of −0.4 eV and −0.3 eV, respectively. In each of the diagrams, three different partial pressures of water vapor are recorded, at a fixed partial pressure CO₂ (0.4 mbar). It can be seen here that the occupancy (load/rel.) also depends heavily on the partial water pressure.

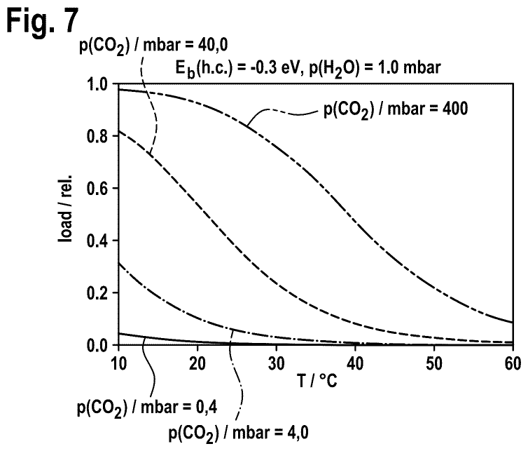

FIG. 7 shows, in a complementary manner, how occupancy (load/rel.) at a fixed partial water pressure changes with temperature T when there are different partial CO₂ pressures. This corresponds qualitatively to a law of mass action (shift of the equilibrium by increasing the concentration of at least one reaction partner).

The particularities of the adsorption processes and the resulting equations of the model described to this extent compared to conventional chemical reactions together with the law of mass action are the non-monotonic process of entropy depending on the occupancy of the sorbent:
a sorbent completely occupied by an adsorbent type and a completely empty sorbent each have an entropy of zero (there is only one microstate corresponding to the macrostate)
the state for maximum entropy in the sorbent is in the uniform distribution of all adsorbents AS with mᵢ=M/k and αᵢ=1/k, respectively.
the derivation of the entropy after occupancy becomes infinitely positive/negative at the edges of the calculated occupancies (limit value considerations mᵢ→0 or mᵢ→M). The sorbent resists exactly complete loading/emptying much more than a partially filled sorbent. Therefore, isobars at very low and very high temperatures T will be horizontal and will have a steep gradient in a temperature window of only a few tens of Kelvins.

With the same formalism, transitional conditions or internal degrees of freedom (e.g. vibrations of an adsorbed molecule group) can also be easily integrated if this state differs from the base level by an energy amount ΔE. Any such additional state may be formally considered as another (k+1)th adsorbent AS by using the same stoichiometry coefficients as for the base state but with an ΔE attenuated binding energy

$$- {E}_{b \_ k + 1} = - {E}_{b \_ k} Δ E .$$

The model from the last section does not yet cover all cases as known for CO₂ adsorption on amines, despite its generality. The adsorbent AS “ammonium carbamate” may also be formed on primary amines. A CO₂ molecule is thereby taken up at two adjacent amine groups (receptors), whereby the distances between the amine groups may not be too great in this case. For thermodynamic modeling, the assumption from the final section (all receptors are the same type) must be modified. It is necessary to take into account the additional spatial proximity of the receptors to one other.

In the following, the case is considered that only the two adsorbents AS “hydronium carbamate” and “ammonium carbamate” can form in competition with each other and in competition with unfilled receptors.

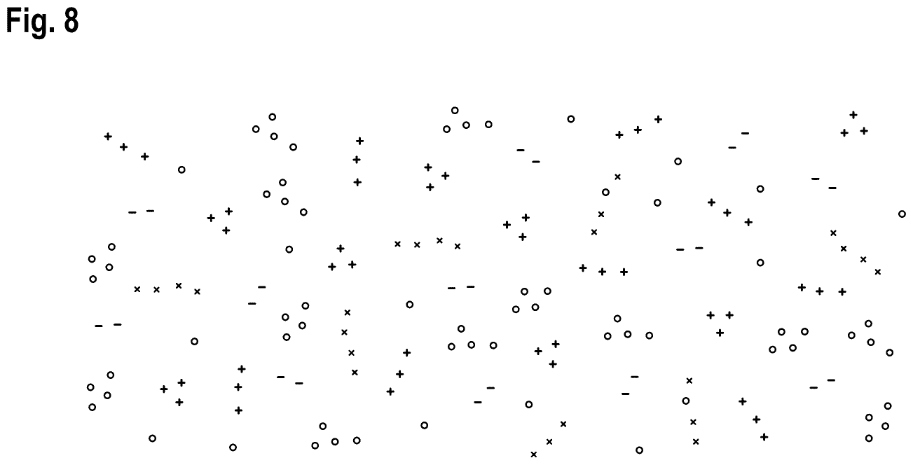

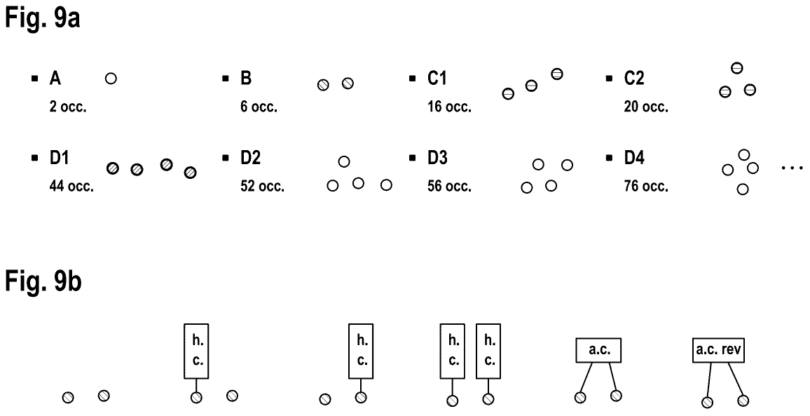

FIG. 8 shows the combining of receptors in clusters. In a single cluster, adjacent receptors are combined when the distances between them are compatible with pairwise adsorption. A cluster may consist of 1, 2, 3, . . . receptor objects. The single, isolated receptor (type A) is a special case if there is no neighbor for pair formation; no ammonium carbamate formation can take place on it, only a hydronium carbamate formation or maintaining the empty position. However, the number of receptors in a cluster is not sufficient for categorization, as shown in FIG. 9a. For example, if there are three or more receptors in a cluster, the receptors can be arranged as a chain, as a triangle, or as a pyramid, for which categories must be defined (C₁, C₂) in each case.

The total sorbent can thus be modeled as a weighted sum of the individual cluster categories. If no further details are known about the structure of the sorbent, then in practice models for different cluster frequencies are calculated and the weightings must be determined as fit parameters.

It is particularly advantageous that the same calculation method as for a single receptor can be used for each cluster category. To do so, formally all of the adsorbent configurations must be considered to be another adsorbent AS having its own binding energy. This is shown using the example of cluster type B in FIG. 9b. All contemplated combinations of how empty spaces and adsorbents AS could be distributed are considered: hydronium carbamate (occupies a receptor with H₂O+CO₂) and ammonium carbamate (occupies two receptors with a CO₂), wherein a mirror asymmetry was also taken into account (a.c. rev)). For cluster type B, there are six adsorption configurations. The competition from these six adsorption configurations is calculated according to the formalism from the last section for p(CO₂), p(H₂O) and temperature T, wherein the stoichiometries for H₂O and CO₂ are considered. If the proportions of the individual occupancies α₀ to α₅ are known, then the equivalents for ammonium carbamate, hydronium carbamate and for H₂O and CO₂ equivalents can be balanced as the overall average in further steps.

However, when establishing a cluster model, it must be noted that the quantity of all contemplated cluster adsorbent occupancies rapidly increases with the number of individual receptors. While there are only six configurations for type “B” (referred to as “occ.” in FIG. 9a), there can already be forty-four to seventy-six configurations for the clusters having four receptors (types “D₁” to “D₄”) (=formal adsorbents AS). Simulations have shown that different cluster types can also demonstrate similar behaviors. It may be sufficient to limit oneself to a small number of cluster types and use fewer weighting factors (to be determined from experiments) for this purpose.

In addition, it is mentioned that it is additionally conceivable to combine the theoretical model of loading, which is calculable as described in a fine grid, with a data-driven model of the reaction rates.

The method described thus far can be altered or modified in many ways without deviating from the idea of the disclosure. This consists of a model in which chemical and physical parameters of the at least one storage substance on the one hand as well as additional weather data WD of a carrier medium are taken into consideration for calculating the amounts of adsorption AS of sorbents S in at least one storage substance.

1. A method comprising:
determining an amount of adsorption of sorbents in at least one storage substance by:
taking into account both physical and chemical parameters of the at least one storage substance and taking into account a theoretical and/or data-based adsorption behavior of the at least one storage substance; and
determining the amount of adsorption of the sorbents based on a plurality of physical data of a carrier medium associated with different points in time and/or periods of time, wherein the physical data comprises at least one temperature of the carrier medium, which contains the sorbents and chemically reacts with the at least one storage substance, at the different times and/or periods of time.

2. The method according to claim 1, wherein the determining of the amount of adsorption includes considering a pressure of the carrier medium at the different times and/or periods of time.

3. The method according to claim 1 wherein:
the determining of the amount of adsorption includes determining the amount of adsorption of CO₂ as one of the sorbents, and
the physical data of the carrier medium is weather data of a location, the weather data comprising at least air temperature.

4. The method according to claim 3, wherein the weather data is historical weather data and/or forecasted weather data.

5. The method according to claim 4, the weather data evaluated and/or forecasted at particular times and/or time periods is used to determine the amount of adsorption.

6. The method according to claim 3, wherein location-specific weather data (WD) is used to compare different locations for possible amounts of adsorption.

7. The method according to claim 6, wherein fixed physical and chemical parameters of the at least one storage substance are used for the comparison.

8. The method according to claim 1, wherein:
the determining of the amount of adsorption of the sorbent is based on statistical physics, and includes calculating proportions of different possible adsorbents of the at least one storage substance and empty spaces that are distributed across like receptors.

9. The method according to claim 8, wherein, for each possible adsorbent of the at least one storage substance, stoichiometry of all involved sorbents is considered, entropy of the receptors is calculated, and chemical potentials of the storage substance for each adsorbent is linked to the chemical potentials of gases involved in the process.

10. The method according to claim 8, wherein sorbents are bound by more than one receptor of the adsorbents of the at least one storage substance.

11. The method according to claim 10, wherein distances between and spatial arrangement of the receptors are combined into individual cluster types and the cluster types are considered as receptors.

12. A device comprising:
a data processing system configured to perform the method according to claim 1.

13. A computer program product comprising:
a data program or data storage medium comprising commands that cause the device of claim 12 to perform the determining of the amount of adsorption of the sorbents in the at least one storage substance.
